(19) United States
(12) Patent Application Publication
(10) Pub. No.: US 2017/0153214 A1
(43) Pub. Date: Jun. 1, 2017

(54) OPTICAL SENSING MODULE

(71) Applicant: Industrial Technology Research Institute, Hsinchu (TW)

(72) Inventors: Chin-Hung Wang, Hsinchu City (TW); Cheng-Ta Ko, Taipei City (TW); Sheng-Shu Yang, Hsinchu City (TW)

(21) Appl. No.: 14/952,924

(22) Filed: Nov. 26, 2015

(51) Int. Cl. G01N 33/00; G01N 21/27; G01N 21/25

(52) U.S. Cl. CPC G01N 33/0062; G01N 21/255; G01N 21/27; G01N 33/0027; G01N 33/0013; G01N 2201/061

(57) ABSTRACT

An optical sensing module, adapted to sense a characteristic of an object by a sensing beam, comprises a carrying substrate, a transparent cover having a reflective surface thereon, a side wall, an optical grating, and an optical sensor. The reflective surface has a light-transmissive opening that exposes a part of the transparent cover. The side wall is disposed around the carrying substrate and is located between the carrying substrate and the transparent cover. The optical grating is disposed on the carrying substrate and a position of the optical grating corresponds to the light-transmissive opening. The optical sensor is disposed on the carrying substrate and is located at a side of the optical grating, wherein the carrying substrate, the side wall and the transparent cover form a vacuum chamber. The optical grating and the optical sensor are disposed in the vacuum chamber.

### TECHNICAL FIELD

The technical field relates to an optical sensing module.

### BACKGROUND

With the industrial development and the urban modernization, a large amount of pollutants in the atmosphere increases quickly and causes air quality deterioration, and endangering human health.

Therefore, the gas sensor is developed and used to sense the atmosphere pollutants. For example, the general gas sensor has specific metal oxide, and the specific metal oxide chemically reacts with air pollutants to determine the feature of the air pollutants. However, the metal oxide of the gas sensor could only react with specific pollutants, it could not react with other kind of gas. In other words, the specific metal oxide of the gas sensor could only detect specific gas pollutants one to one, and it's not convenient for the user. On the other hand, the metal oxide is damp easily and deterioration, so the gas sensor could not perform the detection of air pollutions.

Thus, the optical gas sensor is developed to solve the problems. The principle is after the sensing beam passing the air pollutions, the optical gas sensor receives the sensing beam and gets the material characteristics of the air pollutants. The light transmitting path of the gas sensor is long, and the light intensity is easily decayed. Also, the volume of the module is large, and it is not portable easily. It has no characteristic of real-time monitoring. It's important for the researcher seeking to break through these issues.

### SUMMARY

According to an embodiment of the disclosure, an optical sensing module is adapted to sense a characteristic of an object by a sensing beam. The optical sensing module comprises a carrying substrate, a transparent cover having a reflective surface thereon, a side wall, an optical grating, and an optical sensor. The reflective surface has a light-transmissive opening that exposes a part of the transparent cover. The side wall is disposed around the carrying substrate and is located between the carrying substrate and the transparent cover. The optical grating is disposed on the carrying substrate and a position of the optical grating corresponds to the light-transmissive opening. The optical sensor is disposed on the carrying substrate and is located at a side of the optical grating, wherein the carrying substrate, the side wall and the transparent cover form a vacuum chamber. The optical grating and the optical sensor are disposed in the vacuum chamber.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1A is a schematic cross-sectional view of an optical sensing module according to an embodiment of the disclosure.

FIG. 1B is a partial schematic cross-sectional view of the optical sensing module of FIG. 1A.

FIG. 1C to FIG. 1F are partial schematic cross-sectional views of the optical sensing module according to embodiments of the disclosure.

FIG. 2A to FIG. 2F illustrate a manufacturing flow of the optical sensing module according to an embodiment of the disclosure.

### DETAILED DESCRIPTION OF DISCLOSED EMBODIMENTS

To elaborate a plurality of embodiments of the optical sensing module of the disclosure in detail, the optical sensing module is regarded in the X, Y, and Z axes constructed in the space. X axis direction is along the horizontal direction, the Z axis direction is perpendicular to the X axis direction and extended along the vertical direction, and the Y axis direction is perpendicular to the X axis direction and perpendicular to the Z axis direction.

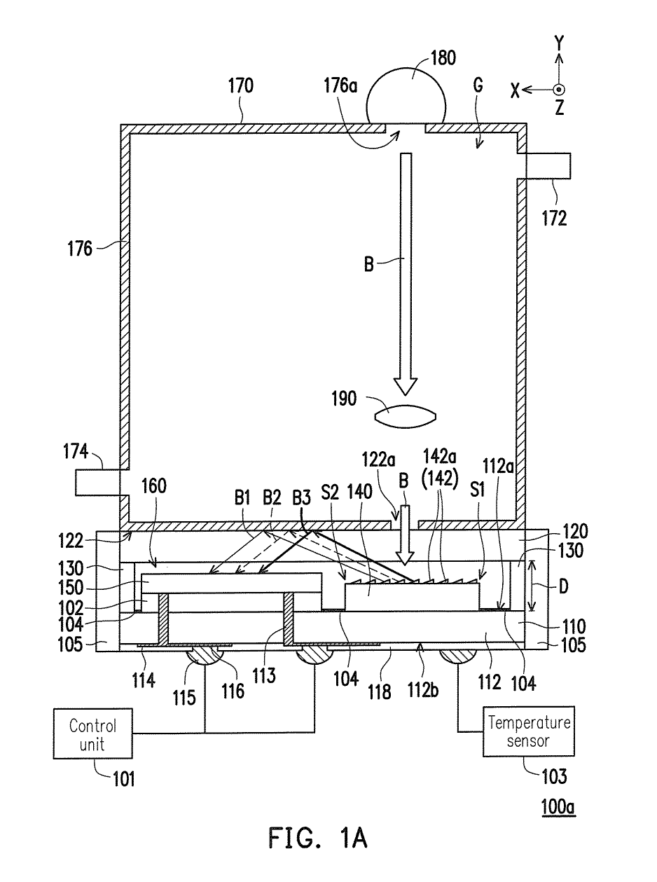

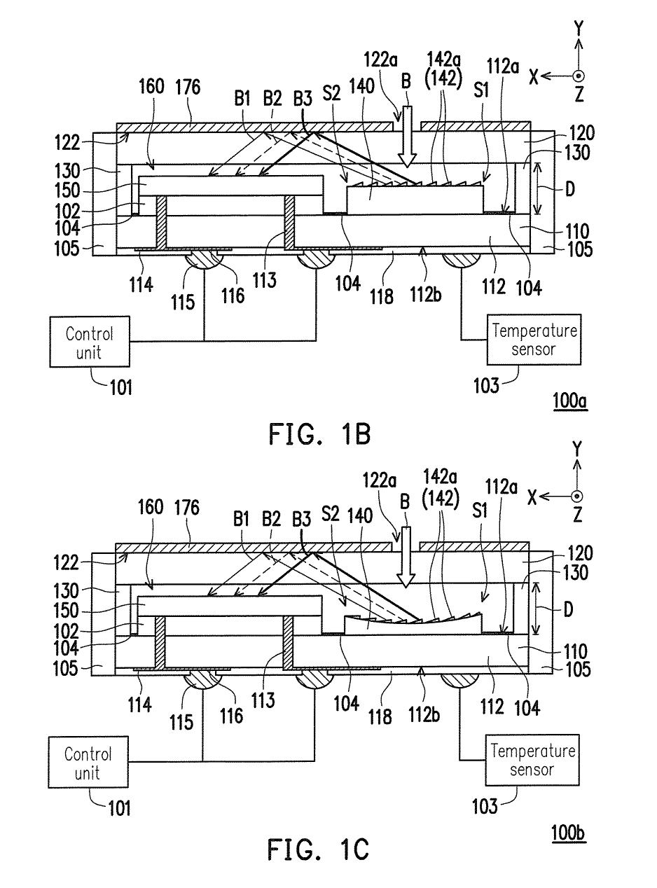

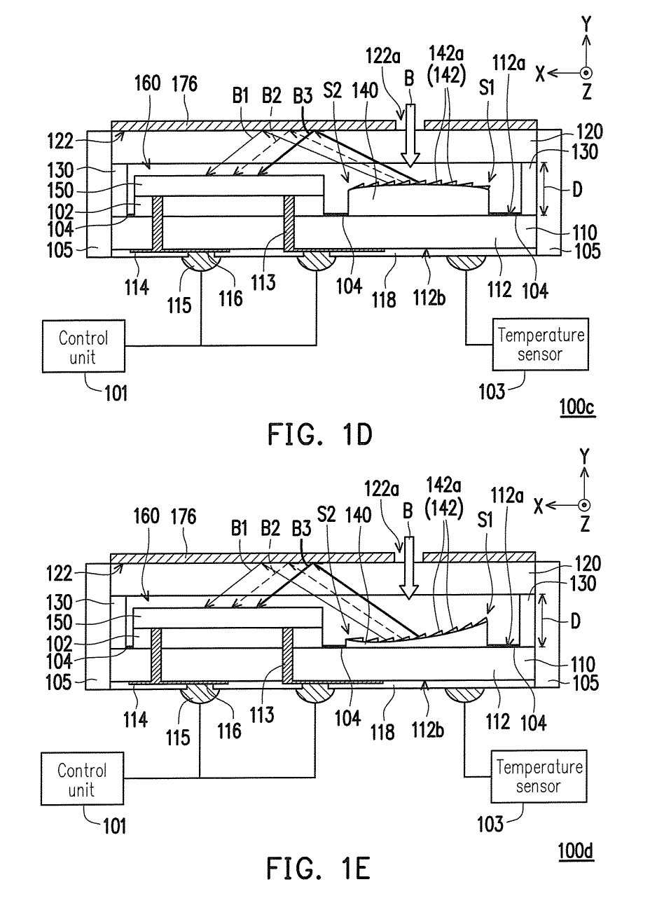

FIG. 1A is a schematic cross-sectional view of an optical sensing module 100a according to an embodiment of the disclosure, wherein the characteristic of an object G is sensed by a sensing beam. Referring to FIG. 1A, the optical sensing module 100a comprises a carrying substrate 110, a transparent cover 120, a side wall 130, an optical grating 140 and an optical sensor 150. A reflective surface 122 is disposed on the transparent cover 120, the reflective surface 122 has a light-transmissive opening 122a, and the light-transmissive opening 122a exposes a part of the transparent cover 120. The side wall 130 is disposed around the carrying substrate 110, and is located between the carrying substrate 110 and the transparent cover 120. The optical grating 140 is disposed on the carrying substrate 110, and the position of the optical grating 140 corresponds to the light-transmissive opening 122a. The optical sensor 150 is disposed on the carrying substrate 110, and located at a side of the optical grating 140, wherein the carrying substrate 110, the side wall 130 and the transparent cover 120 form a vacuum chamber, and the optical grating 140 and the optical sensor 150 are disposed in the vacuum chamber 160.

The material of transparent cover 120 may be, but not limited to glass or other materials of high gas tightness and high light transmittance material. The material of the carrying substrate 110 may be, but not limited to glass, silicon or other materials of high gas tightness. Both the transparent cover 120 and the carrying substrate 110 have a characteristic of high gas tightness, therefore, it is not easy for the external gas or liquid to enter the vacuum chamber 160. This may make the pressure of the vacuum chamber 160 keeps a fixed range. It may also prevent the optical sensor 150 from the pollutions of the impurities or the particles.

In the disclosure, the meaning of the term ‘vacuum’ in the vacuum chamber 160 indicates a ‘substantial vacuum’, instead of an ‘absolute vacuum’. An exemplar of an absolute vacuum for a vacuum chamber is the vacuum chamber has a gas pressure of 0 torr. While the ‘vacuum’ for the vacuum chamber 160 in the disclosure is relative to an outer environment. In an exemplary manufacturing process for the optical sensing module 100a, there is remaining gas in the vacuum chamber 160 of the optical sensing module 100a. In the embodiments of the disclosure, a gas pressure of the vacuum chamber 160 has a range that goes from 1×10⁻² torr to 1×10⁻⁷ torr

In an embodiment, the side wall 130 may be, but not limited to a gas adsorption layer, wherein the material of the gas adsorption layer is selected form the group consisting of Copper (Cu), Aluminium (Al), Vanadium (V), Zirconium (Zr), Cobalt (Co) or a combination thereof, but a true scope of the disclosure is not limited thereto. In other words, the material of the gas adsorption layer may be at least one metal or a metal alloy with chemical reactions. Also, the gas adsorption layer may have chemical reactions with some gases in the vacuum chamber 160. The followings are some exemplars, wherein, the side wall 130 (gas adsorption layer) may have different chemical reactions with different gases in the vacuum chamber 160.

GM+O₂→GMO;

GM+N₂→GMN;

GM+CO₂→CO+GMO→GMC+GMO;

GM+CO→GMC+GMO;

GM+H₂O→H+GMO→GMO+H;

GM+H₂→GM+H;

GM+Hydrocarbons, (CₓHₓ)→GMC+H;

GM+inert gases→No Reaction

Wherein the GM in the above-mentioned chemical equations represents the side wall 130 (for example, a gas adsorption layer). It may be seen from these chemical equations that the side wall 130 (for example, a gas adsorption layer) may further have at least one chemical reaction with one or more gases in the vacuum chamber 160, and which may form chemical compounds having a lower vapor pressure on a surface of side wall 130 (gas adsorption layer). Accordingly, the side wall 130 (for example, a gas adsorption layer) may continuously have chemical reactions with some gases in the vacuum chamber 160. Therefore, the gas pressure of the vacuum chamber 160 reaches a dynamic balance, and has a range of lower pressure maintained in the vacuum chamber 160 relative to the outer environment. As aforementioned, the side wall 130 (for example, a gas adsorption layer) may have chemical reactions with gases in the vacuum chamber 160. Therefore, after the sensing beam B that penetrates the light-transmissive opening 122a into the vacuum chamber 160, there is less probability for the sensing beam to pass through the gas remaining in the vacuum chamber 160. This may avoid the gas remaining in the vacuum chamber 160 to affect the overall sensing results. Thus, the sensing results according to the embodiment of the optical sensing module 100a may have a higher accuracy.

The following paragraph provides a comparison for an optical sensing module of with the gas adsorption function versus without the gas adsorption function. The following Table 1 provides gas properties in a vacuum chamber of a comparative embodiment (which is the side wall has no gas adsorption function) of an optical sensing module and a present embodiment (which is the side wall has a gas adsorption function, for example, a gas adsorption layer) of the optical sensing module 100a in the disclosure.

TABLE I

| | The present embodiment | | Comparative embodiment | |
|---|---|---|---|---|
| | | Percentage of | | Percentage of |
| | | gas in the | | gas in the |
| | Pressure | chamber | pressure | chamber |
| Gas type | (mbar) | (%) | (mbar) | (%) |
| H₂ | 0.00E+00 | 0.00% | 3.04E−01 | 6.38% |
| He | 2.18E−03 | 95.76% | 2.16E−03 | 0.05% |
| CO | 0.00E+00 | 0.00% | 3.18E−01 | 6.67% |
| N₂ | 0.00E+00 | 0.00% | 3.41E+00 | 71.53% |
| CH₄ | 0.00E+00 | 0.00% | 4.64E−01 | 9.72% |
| H₂O | 0.00E+00 | 0.00% | 0.00E+00 | 0.00% |
| O₂ | 0.00E+00 | 0.00% | 3.91E−04 | 0.01% |
| C₂H₆ | 0.00E+00 | 0.00% | 6.45E−03 | 0.14% |
| C₃H₈ | 0.00E+00 | 0.00% | 6.88E−03 | 0.14% |
| Ar | 9.66E−05 | 4.24% | 1.47E−04 | 0.00% |
| CO₂ | 0.00E+00 | 0.00% | 2.56E−01 | 5.37% |
| Kr | 0.00E+00 | 0.00% | 0.00E+00 | 0.00% |
| Pressure | 2.28E−03 | 100.00% | 4.77E+00 | 100.00% |
| of the | | | | |
| chamber | | | | |

It may be seen from the Table 1, in the present embodiment of the optical sensing module 100a, the side wall 130 (for example, a gas adsorption layer) has gas adsorption function, and the vacuum chamber 160 of the present embodiment has a lower gas pressure. Also, the side wall 130 may further have chemical reactions with the gas of the Hydrocarbon (for instance, CH₄, C₂H₆ or C₃H₈) remaining in the vacuum chamber 160, and the sensing results are not affected by the remaining gas. It may also be seen from the Table 1 that most of the remaining gases in the vacuum chamber 160 are inert gases such as Helium (He) and Argon (Ar). It is known that the chemical properties of He and Ar are stable, and He and Ar have no chemical reaction with the sensing beam B, therefore, the sensing results according to the present embodiment of the optical sensing module 100a have a higher accuracy.

In an embodiment of the disclosure, a distance D between the transparent cover 120 and the carrying substrate 110 may be less than 300 μm. If the distance D is too long, the light intensity of the sensing beam B entering the vacuum chamber 160 is decayed and the sensing result is affected.

Referring back to the embodiment of FIG. 1A, the optical sensing module 100a senses a characteristic of the object G by the sensing beam B. The optical sensing module 100a further comprises a transparent cavity 170, a light source and a focusing lens 190. The transparent cavity 170 comprises an inlet 172 and an outlet 174, wherein the inlet 172 makes the object G entering the transparent cavity 170 and the outlet 174 makes the object G exit the transparent cavity 170. The light source 180 is disposed on an outer surface of the transparent cavity 170, and the sensing beam B penetrates the transparent cavity 170 into the light-transmissive opening 122a. In an embodiment, light-transmissive opening 122a may be, but not limited to a slit. The focusing lens 190 is disposed on the transmission path of the sensing beam B, and the focusing lens 190 is disposed between the light source 180 and the light-transmissive opening 122a. The focusing lens 190 is used to focus the sensing beam B, avoiding that the light path of the sensing beam B is too long so the light intensity decays and affecting the sensing results of the optical sensing module 100a.

The state of the object G is not limited in the disclosure. For example, the object G may be liquid to be tested. Therefore, the optical sensing module 100a in the disclosure may detect the to-be-tested gases in the atmosphere, such as pollutants in the atmosphere. It may also sense different components in the liquid to be tested. In other words, the optical sensing module 100a in the disclosure may sense all the to-be-tested materials or articles allowing light to pass through.

In the embodiment, the transparent cavity 170 further includes a reflective layer 176 disposed on an inner surface of the light-transmissive cavity 170, and the reflective layer 176 has an opening 176a. The opening 176a makes the sensing beam B pass through the light-transmissive cavity 170. The material of the reflective layer 176 may be, but not limited to metals or other materials of a high light reflectance. The reflective layer 176 may avoid the external light beam entering the light-transmissive cavity 170 and influencing the sensing results of the optical sensing module 100a. The reflective layer 176 may also allow the partial sensing beam B without entering the light-transmissive opening 122a reflecting one to multiple times and then entering the light-transmissive opening 122a. In other words, the disposition of the reflective layer 176 may raise the probability of entering the light-transmissive opening 122a for the sensing beam B.

In the disclosure, the term “light-transmissive” of the light-transmissive opening 122a and the light-transmissive cavity 170 is relative to the wavelength of the sensing beam B. The light emitting device of the light source 180 may be, but not limited to the Light Emitting Diode, Laser or any other type of light emitting device. The wavelength of the sensing beam B emitting form the light source 180 may be, but not limited to the wavelength of visible light, infrared light, ultraviolet light or other different wavelengths. According to different wavelengths emitting from the sensing beam B, the optical sensor 150 may be Visible light sensor, Infrared light sensor, Ultraviolet light sensor or other sensor sensing different wavelength, as long as the sensing beam B may be detected by the optical sensor 150. On the other hand, the type of the optical sensor 150 may be, but not limited to Complementary Metal Oxide Semiconductor, Charge Coupled Device and other solid state optical sensor. The optical sensor 150 may be, but not limited to a one-dimensional optical sensor or a two-dimensional optical sensor.

FIG. 1B is a partial schematic cross-sectional view of the optical sensing module of FIG. 1A, which further illustrates the transmission path in the vacuum chamber 160 of the sensing beam B of the present embodiment. Please refer to FIG. 1B, in the embodiment, the sensing beam B penetrates the light-transmissive opening 122a (slit) into the vacuum chamber 160 and then arrives at the optical grating 140. The optical grating 140 diffracts the sensing beam B in the vacuum chamber 160 and different wavelengths of diffracted sensing beams B1, B2, B3 are formed (the three diffraction light beams in FIG. 1A and FIG. 1B are illustrated schematically), and the diffracted sensing beams B1, B2, B3 further transmits to the reflective surface 122, and the diffracted sensing beam (diffracted sensing beams B1, B2, B3) are reflected to the optical sensor 150 by the reflective surface 122. Then, spectrum is formed by at least a part of diffracted sensing beams B1, B2, B3 transmitting to the optical sensor 150. In detail, the spectrum detected by the optical sensor 150 is the absorption spectrum of the object G. In the embodiment, the characteristic of the object G detected by the sensing beam B is the absorption spectrum of the object G. In other words, an exemplary characteristic of the object G is the absorption spectrum of the object G for instance. In other embodiments, the spectrum may be, but not limited to the emission spectrum, photo-reflectance spectrum, photoluminescence spectrum, Raman spectrum, transmission spectrum, reflection spectrum or other kinds of spectrum. The characteristics of the object G are not only the spectrum itself, they may be different characteristics of the object G gained by the spectrum or a characteristic different from the spectrum, but the scope of the disclosure is not limited thereto.

In an embodiment, the optical sensor 150 and the optical grating 140 are disposed on the first surface 112a, the position of the optical grating 140 corresponds to the light-transmissive opening 122a, and the optical grating 140 is located next to the optical sensor 150. The sensing beam B penetrates the light-transmissive opening 122a into the vacuum chamber 160 and is reflected to the optical sensor 150 through the above-mentioned transmitting path. Thus the sensing beam B of the disclosure may transmit to the optical sensor 150 by a shorter transmitting path, and the optical sensing module 100a may have effects of smaller volume and portable use, and this facilitates to achieve the real-time monitoring. In addition, it may shorten the transmitting path and improve the accuracy of the sensing results.

In an embodiment, of the optical sensor 150 comprises a plurality of pixels (not shown), and the plurality of pixels are arranged in an array, for example, an M×N pixel array, wherein M and N are positive integers and M≠N. Each pixel further includes a Photo Diode (not shown) and a signal output end (not shown). The Photo Diode may transform the light signal to the electric signal, and output the electric signal to the output end. When the diffracted sensing beams B1, B2, B3 transmit to different positions of the optical sensor 150, each pixel may sense the corresponding light intensities of different wavelengths of the diffracted sensing beams (B1, B2, B3). The Photo diode may emit different electric signals corresponding to different light intensities, respectively, and each of the electric signals is outputted to the output end. Thus, the optical sensor 150 detects the luminosity information of the sensing beam B passing through the object G. In detail, the optical sensor 150 detects the luminosity information of the sensing beam B passing through the object G according to the light intensity of each of diffracted sensing beams (B1, B2, B3).

In the embodiment, the optical sensing module 100a further comprises an Analog-to-Digital Converter (ADC) 102, wherein the Analog-to-Digital Converter 102 is disposed between the carrying substrate 110 and the optical sensor 150. The optical sensor 150 may transform the detected luminosity information to the analog signal and output to the Analog-to-Digital Converter 102. The Analog-to-Digital Converter 102 performs a Digital Signal Processing on the analog signal (luminosity information), i.e. the luminosity information is transformed from an analog signal to a digital signal.

Please refer back to the embodiment of FIG. 1A. The carrying substrate 110 may further include a substrate 112, a circuit layer 114, a plurality of pads 116, a passivation layer 118, and a plurality of conducting vias 113. The substrate 110 has a first surface 112a and a second surface 112b substantially opposite to the first surface 112a. To simplify the description, the known internal connecting structure of the substrate 112 is not shown. The substrate 112 may be, but not limited to a single-side circuit board, a double-side circuit board or a multi-layer circuit board. The side wall 130, the optical grating 140 and the optical sensor 150 are disposed on the first surface 112a. In other words, the optical grating 140 and the optical sensor 150 are disposed in the vacuum chamber 160 defined by the first surface 112a, side wall 130 and the carrying substrate 110. A circuit layer 114 is disposed on the second surface 112b. A plurality of pads 116 is electrically connected to the circuit layer 114. A passivation layer 118 is disposed on the circuit layer 114 and the plurality of pads 116 are exposed, wherein the material of passivation layer 118 may be, but not limited to an oxide insulating material or a macromolecule insulating material. Each of conducting vias 113 penetrates the substrate 112, and one end of the conducting via 113 connects the optical sensor, the other end of the conducting via 113 connects the circuit layer 114. In addition, the carrying substrate 110 further comprises a plurality of bumps 115, wherein the plurality of bumps 115 are disposed on the plurality of pads 116, respectively.

Each of pads 116 may be an input/output pad of an integrated circuit chip. And each of bumps 115 is an input/output bump and is disposed on a corresponding pad of pads 116. In addition, the material of the bumps 115 is selected from at least one of the group consisting of Stannum (Sn), Silver (Ag), Copper (Cu) or the alloy thereof, or other unleaded or other lead-free alloy, to avoid causing the environmental pollution, but the scope of the disclosure is not limited thereto. In the embodiment, the optical sensing module 100a may further include a control unit 101 storing the comparison information. The control unit 101 may be, but not limited to a calculator, a Micro Controller Unit, a Central Processing Unit, or a programmable Microprocessor Digital Signal Processor, a programmable controller, Application Specific Integrated Circuits, a programmable Logic Device or any similar device. The control unit 101 is electrically connected to the optical sensor 150 by the circuit layer 114, the pads 116 and the bumps 115. The control unit 101 receives the luminosity information transmitted by the optical sensor 150. In an embodiment, the control unit 101 receives the luminosity info nation (digital signal) transformed by the Analog-to-Digital Converter 102. The control unit 101 compares the luminosity information and the comparison information, wherein the luminosity information is determined by the sensing beam B passing through the object G. The comparison flow of the luminosity information and the comparison information will be illustrated in the followings.

In an embodiment, the optical sensing module 100a further comprises a temperature sensor 103. Temperature sensor 103 is electrically connected to the control unit 101 by the circuit layer 114, the pads 116 and the bumps 115. The temperature sensor 103 senses a temperature information of the optical sensing module 100a, and the temperature information is the temperature of the optical sensing module 100a. The control unit 101 determines the comparison information according to the temperature information.

In an embodiment, the comparison information stored in the control unit 101 includes absorption wavelengths corresponding to different chemical bonding types, or different absorption wave bands respectively corresponding to different gases. These absorption wavelengths corresponding to different bonding types, and different absorption wave bands respectively corresponding to different gases are functions of temperature. Therefore, the temperature of the optical sensing module 100a is known after the control unit 101 receives the temperature information, and the comparison information corresponding to the temperature is selected according to the temperature of the optical sensing module 100a. When the control unit 101 receives the luminosity information, the control unit 101 may compare the luminosity information with the selected comparison information, to determine the characteristic of object G. The following table II and table III are some exemplary comparison information at a room temperature T (=300K) of the environmental temperature.

TABLE II

| Gas type | Bonding type | Absorption wavelength (μm) |
|---|---|---|
| Fuel gas | —C—H | 3.5 |
| | —CH₂ | 6.89 |
| | CH₄ | 7.6 |
| Olefin | ═C—H | 3.33 |
| | C═C | 6.25 |
| Alkyne | ≡C—H | 3.03 |
| | C≡C | 4.76 |
| Benzene | ═C—H | 3.30 |
| | C═C | 6.77 |
| CO₂ | C═O | 4.3 |
| CO | C≡O | 4.67 |
| Aldehyde | C═O | 5.91 |

TABLE III

| Gas type | Absorption wavelength (μm) |
|---|---|
| N₂ | <0.1 |
| O₂ | <0.245 |
| O₃ | 0.17-0.35 |
| | 0.45-0.75 |
| H₂O | <0.21 |
| | 0.6-0.72 |
| H₂O₂ | <0.35 |
| NO₂ | <0.6 |
| N₂O | <0.24 |
| NO₃ | 0.41-0.67 |
| HONO | <0.4 |
| HNO₃ | <0.33 |
| CH₃Br | <0.26 |
| CFCl₃ | <0.23 |
| HCNO | 0.25-0.36 |

The information disclosed here is for example, the comparison information may be other kinds of gas not mentioned form the above-mentioned tables, such as the absorption wavelength of the gas, solid, liquid or different bonding types of chemical bonding, but the scope of the disclosure is not limited thereto.

The partial sensing beam B in the vacuum chamber 160 may not be reflected to the optical sensor 150 by the reflective surface 122 directly. And the partial sensing beam B may firstly transmit to the first surface 112a uncovered by the side wall 130, the optical grating 140 and the optical sensor 150, then is reflected to the reflective surface 122 and further reflected to the optical sensor 150 through the reflective surface 122.

Therefore, the sensing results of the optical sensing module 100a may be affected. In an embodiment, the optical sensing module 100a further comprises a light-absorbing layer 104 disposed on the first surface 112a uncovered by the side wall 130, the optical grating 140 and the optical sensor 150. The light-absorbing layer 104 is adapted to absorb partial sensing beam B. Thus, the light-absorbing layer 104 may reduce the number of reflecting the sensing beam B, and the sensing result of the optical sensing module 100a is more accurate.

In an embodiment, the optical sensing module 100a further comprises a shading member 105 disposed around the periphery of the transparent cover 120, the side wall 130 and the carrying substrate 110. The shading member 105 is a housing made of plastic, the housing is used to fix the transparent cover 120, the side wall 130 and the carrying substrate 110. The shading member 105 is used to shelter the periphery of the transparent cover 120, the side wall 130 and the carrying substrate 110, and avoid the environmental beam penetrating the vacuum chamber 160 to affect the sensing result of the optical sensing module 100a. The shading member 105 may be, but not limited to metal foil, and the metal foil may be deposited around the periphery of the transparent cover 120, the side wall 130 and the carrying substrate 110 by a vapor deposition scheme or a sputtering deposition scheme. In the disclosure, a member having a shading function may be used as the shading member 105. In other words, the shading member 105 may be embodied in various forms without being limited to the aforementioned exemplary embodiments.

Referring back to the FIG. 1B, the optical grating 140 may further comprises a diffractive surface 142, a first end S1 and a second end S2. The diffractive surface 142 has a plurality of diffractive structures 142a. A shape of the diffractive structures 142a may be, but not limited to a zigzag or a wavy. The first end S1 is away from the optical sensor 150, and the second end S2 substantially opposite to the first end S1. The second end S2 is adjacent to the optical sensor 150, wherein a distance between the first end S1 and the reflective surface 122 is less than that between the second end S2 and the reflection surface 122. In other words, a macroscopic outline of the diffractive surfaces 142 comprises a plane shown in FIG. 1B, and the diffractive structures 142a are arranged along the plane. In the embodiment, the extending direction of the light-transmissive opening 122a is, for example, along the Z axis, and the extending direction of the diffractive structures 142a is also along the Z axis. It means that both the light-transmissive opening 122a and the diffractive structures 142a have the same extending direction that is substantially along the Z axis. The optical sensing module 100a may further comprise at least one functional device such as calculation module, storage module, communication module, and power module etc., but the scope of the disclosure is not limited thereto.

FIGS. 1C and 1D are partial schematic cross-sectional views of the optical sensing modules 100b and 100c, respectively, according to embodiments of the disclosure. As aforementioned and illustrated in FIG. 1B, the macroscopic outline of the diffractive surfaces 142 of the optical sensing module 100a comprises a plane and the diffractive structures 142a is arranged along the plane. While, the macroscopic outline of the diffractive structures 142a of each of the optical sensing modules 100b and 100c comprises a curved surface, as illustrated in FIGS. 1C and 1D, respectively, and the diffractive structures 142a is arranged along the curved surface. The curved surface of each of the optical sensing modules 100b and 100c towards the transparent cover 120, wherein the curved surface of the optical sensing module 100b toward the transparent cover 120 is a concave surface, while the curved surface of the optical sensing module 100c toward the transparent cover 120 is a convex surface.

FIGS. 1E and 1F are a partial schematic cross-sectional views of the optical sensing modules 100d and 100e, respectively, according to embodiments of the disclosure. As shown in FIGS. 1E and 1F, the distance between the first end S1 and the reflective surface 122 is less than the distance between the second end S2 and the reflection surface 122. In other words, the curved surface mentioned in FIGS. 1C and 1D is inclined from the first end S1 toward the second end S2. The difference between the optical sensing module 100e and the optical sensing module 100c is the same as that between the optical sensing module 100d and the optical sensing module 100b and thus are not be further explained herein.

The followings illustrates a method of sensing an object G, which may be applied to any of the optical sensing modules 100a, 100b, 100c, 100d, and 100e. First, the inlet 172 and the outlet 174 are closed, and the light source 180 emits the sensing beam B; to measure the luminosity information of the transparent cavity 170 without carrying the object G. It means measuring the luminosity information of the gas inside the transparent cavity 170, wherein the measured luminosity information is used for correction. Then, the light source 180 is closed. Also the inlet 172 is opened and the outlet 174 is closed, which allows the object G getting into the transparent cavity 170 via the inlet 172. Then, the switch of the light source 180 is turned on again to let the light source 180 emit the sensing beam B. The sensing beam B passes through the object G, to measure the luminosity information of the object G inside the transparent cavity 170. In the following, the luminosity information of the transparent cavity 170 without carrying the object G is referred as correction information, and the luminosity information of the transparent cavity 170 carrying the object G is referred as luminosity information. There may be some articles (for example, gas or liquid) inside the transparent cavity 170, which may affect the sensing result of the optical sensing module. Therefore, the control unit 101 may correct the luminosity information according to the correction information, before the control unit 101 compares the luminosity information with the comparison information. This avoids the articles (for example, gas or liquid) originally inside the transparent cavity 170 affecting the sensing result. Thus, it may improve the sensing result and improve the accuracy of the optical sensing module of the disclosure. In general, known gas sensors use suction devices to inhale the gas inside the cavity to avoid the gas inside the cavity affecting the sensing result, when they conduct a measurement. Compare to those known gas sensors, the optical sensing module according to the embodiments of the disclosure use less costs and smaller volume.

Therefore, the control unit 101 corrects the luminosity information according to the correction information, then compares the luminosity information with the comparison information. When the control unit 101 detects a weak absorption spectrum of a specific wavelength from the correction, it indicates the object G absorbs the light beam of the specific wavelength of the sensing beam B. For example, if the control unit 101 detects an absorption peak at the wavelength of 4.3 μm, it indicates the object G contains CO₂. Furthermore, the comparison data comprises different absorption coefficients, so the control unit 101 may calculate the concentration of the object G according to the decreasing percentage of the absorption peak. These examples may be considered as exemplary embodiments only, a scope of the disclosure is not limited thereto.

After a period of time, the strength of the sensing beam B emitted from the light source 180 may decay as time pass. When the measurement method using the optical sensing module of the disclosure is applied, that is the luminosity information is corrected according to the correction information, the factors affecting the sensing result of the optical sensing module are avoided. These factors may be, for example, the articles (for example, gas or liquid) originally inside the transparent cavity 170, and the decay of the light strength of the sensing beam from the light source 180.

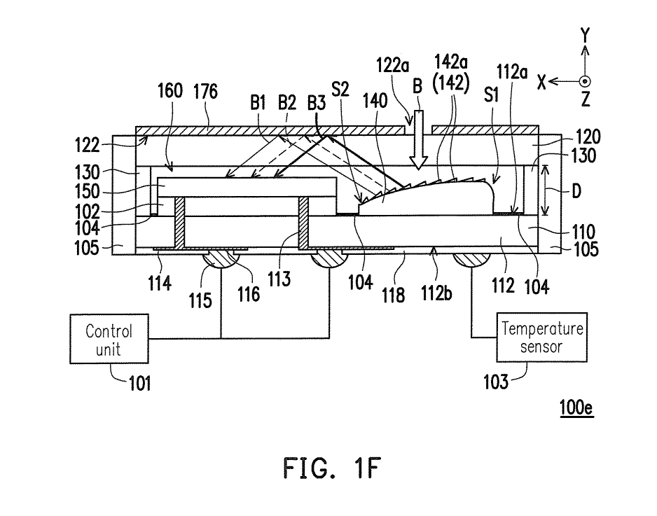

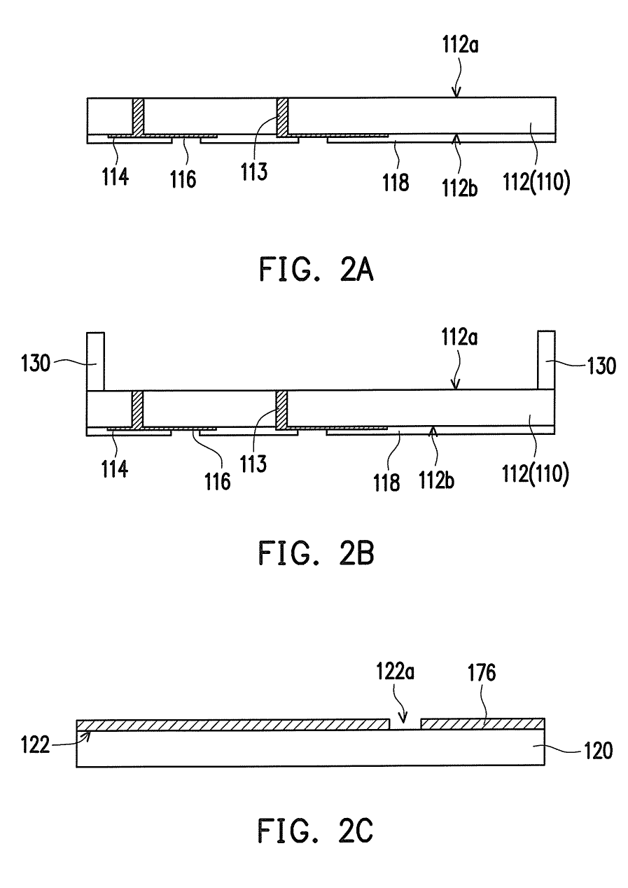

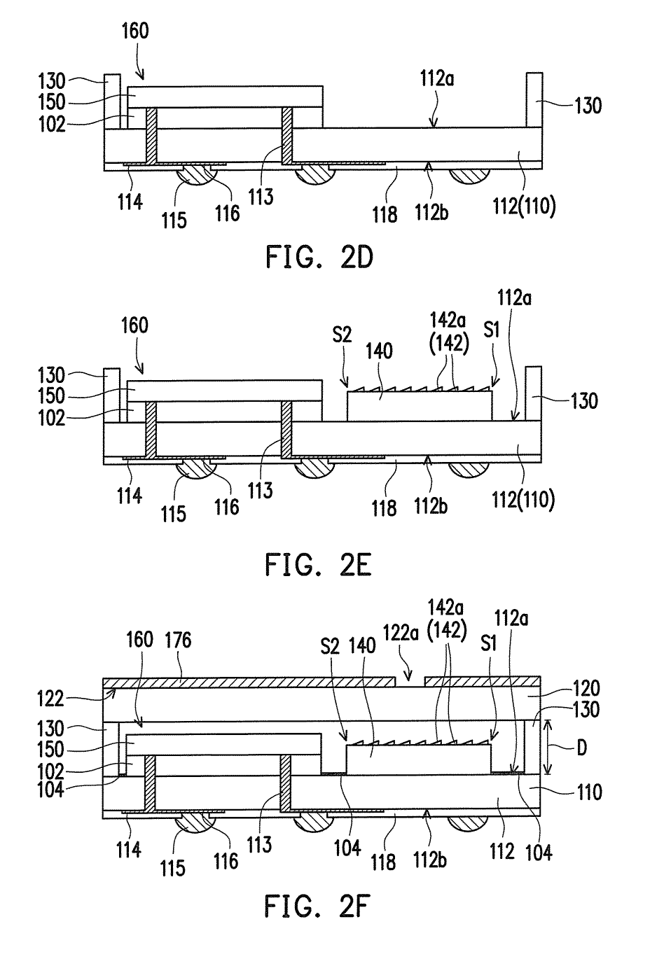

FIG. 2A to FIG. 2F illustrate a manufacturing flow of the optical sensing module according to an embodiment of the disclosure. Exemplary embodiments of the optical sensing module in the disclosure are optical sensing modules 100a, 100b, 100c, 100d, and 100e. Take the optical sensing module 100a as an illustration example. Please refer to FIG. 2A, the carrying substrate 110 is provided. The carrying substrate 110 comprises a substrate 112, a plurality of conducting vias 113, a circuit layer 114, a plurality of pads 116, and a passivation layer 118. In an embodiment, the plurality of conducting vias 113 are formed by a photolithography process on the substrate 112, and the photolithography process defines the positions of the conducting vias 113. Then, an etching process is performed on the positions defining the conducting vias 113, and a plurality of vias are formed by penetrating the substrate 112. The plurality of vias are further filled with metal or metal alloy having a good conductivity to form the conducting vias 113, and the remaining photoresist is removed.

Please refer to FIG. 2A, the substrate 112 comprises a first surface 112a and a second surface 112b substantially opposite to the first surface 112a. The circuit layer 114 is formed on the second surface 112b by a photolithography process. Then, a deposition scheme such as a vapor deposition, a sputtering deposition or other deposition process is used to deposit metal or metal alloy having a good conductivity, for forming the circuit layer 114 on the second surface 112b. The remaining photoresist in the photolithography process is removed after the circuit layer 114 is formed.

In the embodiment, forming the passivation layer 118 may include defining the position of the passivation layer 118 on the second surface 112b by the photolithography process; depositing the passivation layer 118 by a vapor deposition, a sputtering deposition or other deposition process; and removing the remaining photoresist in the photolithography process; wherein the material of the passivation layer 118 may be, but not limited to oxide insulating materials or polymer insulating material. The passivation layer 118 exposes the pads 116 after removing the photoresist.

Please refer to FIG. 2B, the side wall 130 is disposed at the periphery of the carrying substrate 110, wherein the materials of the side wall 130 may include Cu, Al, V, Zr, Co or a combination thereof, and the side wall has the function of sucking gas.

Please refer to FIG. 2C, the transparent cover 120 is provided, wherein the materials of the transparent cover 120 may be, but not limited to glass or other material of high gas tightness and high light transmittance. Then, the position of the light-transmissive opening 122a is defined, a deposition scheme such as a vapor deposition, a sputtering deposition or other deposition process is applied to deposit the reflective layer 176, and the remaining photoresist in the photolithography process is removed.

Please refer to FIG. 2D, a chip-to-wafer technique is employed to connect the optical sensor 150 and the Analog-to-Digital Converter 102 to the first surface 112a, wherein the Analog-to-Digital Converter 102 is disposed between the optical sensor 150 and the carrying substrate 110. One end of each conducting via 113 connects the optical sensor 150, and the other end of each conducting via 113 connects the circuit layer 114. Then, the bumps 115 are formed on the pads 116 respectively, wherein the bumps 115 may be formed by an electro deposition or a welding process. In an embodiment, the materials of the bumps 115 may be selected form at least one of the group consisting of Sn, Ag, Cu, or the alloy thereof; or other lead-free alloy, avoiding the pollution of the environment, but the scope of the disclosure is not limited thereto.

Please refer to FIG. 2E, a chip-to-wafer technique is employed to connect the optical grating 140 having a plurality of diffractive structures 142a to the first surface 112a. The optical grating 140 is located next to the optical sensor 150. The optical sensing module 100a is used here for an illustration exemplar. In other embodiments, instead of the optical grating 140, any optical grating of the illustrated optical sensing modules in the FIG. 1B, FIG. 1C, FIG. 1D, and FIG. 1E may be employed, but the scope of the disclosure is not limited thereto.

Finally, please refer to FIG. 2F, a cap chip bounding technique is employed to connect the transparent cover 120 to the side wall 130, wherein the position of the light-transmissive opening 122a corresponds to the position of the optical grating 140, and the manufacturing process of the optical sensing module 100a is finished.

In summary, a vacuum chamber of an optical sensing module of the disclosed embodiments is formed by a carrying substrate, a side wall and a transparent cover. The optical grating and the optical sensor are disposed inside the vacuum chamber, wherein the pressure of the vacuum chamber has a range that goes from 1×10⁻² to 1×10⁻⁷ torr. This may make the sensing result of the sensing beam is not affected by the remaining gas inside the vacuum chamber. The side wall is may be a gas absorption layer, and the-gas absorption layer may have different chemical reactions with different remaining gases inside the vacuum chamber. The lower vapor pressure of the compound is formed on the surface of the side wall. The pressure in the vacuum chamber may maintain in the aforementioned range. The shading member and the reflective surface may avoid the environmental light penetrating the vacuum chamber to affect the sensing result of the optical sensing module, thus the optical sensing module of the disclosed embodiments may have a higher accuracy. In addition, the optical sensor and the optical grating are disposed on the first surface of the substrate, and the position of optical grating corresponds to the position of the light-transmissive opening. The design of the light path may make the sensing beam having a shorter transmitting path to an optical sensor. Therefore, the optical sensing module may have characteristics of smaller volume, portable use, and real-time monitoring.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. An optical sensing module adapted to sense a characteristic of an object by a sensing beam, comprising:
a carrying substrate;
a transparent cover having a reflective surface thereon, wherein the reflective surface has a light-transmissive opening that exposes a part of the transparent cover;
a side wall, disposed around the carrying substrate and located between the carrying substrate and the transparent cover;
an optical grating disposed on the carrying substrate, wherein a position of the optical grating corresponds to the light-transmissive opening; and
an optical sensor disposed on the carrying substrate and located at a side of the optical grating, wherein the carrying substrate, the side wall and the transparent cover form a vacuum chamber, and the optical grating and the optical sensor are disposed in the vacuum chamber.

2. The optical sensing module as recited in claim 1, wherein the sensing beam penetrates the light-transmissive opening into the vacuum chamber and then arrives at the optical grating, the optical grating diffracts the sensing beam in the vacuum chamber and the diffracted sensing beam is reflected to the optical sensor by the reflective surface.

3. The optical sensing module as recited in claim 1, wherein the optical grating includes a diffractive surface having a plurality of diffractive structures.

4. The optical sensing module as recited in claim 3, wherein a shape of the plurality of diffractive structures is serrated or wavy.

5. The optical sensing module as recited in claim 3, wherein the plurality of diffractive structures are arranged along a curved surface or a plane, and the curved surface is a concave or a convex toward the transparent cover.

6. The optical sensing module as recited in claim 5, wherein the optical grating further includes:
a first end, away from the optical sensor; and
a second end, substantially opposite to the first end, and the second end adjacent to the optical sensor, wherein a distance between the first end and the reflective surface is less than that between the second end and the reflection surface.

7. The optical sensing module as recited in claim 3, wherein an extending direction of the light-transmissive opening is substantially the same as that of the diffractive structures.

8. The optical sensing module as recited in claim 1, wherein the carrying substrate includes:
a substrate including a first surface and a second surface substantially opposite to the first surface, wherein the side wall, the optical grating and the optical sensor are disposed on the first surface;
a circuit layer disposed on the second surface;
a plurality of pads electrically connected to the circuit layer;
a passivation layer disposed on the circuit layer and exposing the pads; and
a plurality of conducting vias penetrating the substrate, wherein each conducting via includes two ends with one end connecting the optical sensor and the other end connecting to the circuit layer.

9. The optical sensing module as recited in claim 8, further including:
a light-absorbing layer disposed on the first surface uncovered by the side wall, the optical grating and the optical sensor.

10. The optical sensing module as recited in claim 8, further including:
a control unit, storing a comparison information and electrically connected to the optical sensor by the circuit layer, wherein the control unit receives a luminosity information transmitted by the optical sensor, and compares the luminosity information with the comparison information, wherein the luminosity information is determined by the sensing beam passing through the object.

11. The optical sensing module as recited in claim 10, wherein the optical sensing module further includes a temperature sensor electrically connected to the control unit by the circuit layer, the temperature sensor senses a temperature information and transmits the temperature information to the control unit, and the control unit determines the comparison information according to the temperature information.

12. The optical sensing module as recited in claim 1, further including:
a light-transmissive cavity having an inlet and an outlet, wherein the inlet and the outlet make the object enter and exit the light-transmissive cavity, respectively;
a light source emitting the sensing beam, wherein the light source is disposed on an outer surface of the light-transmissive cavity, and the sensing beam penetrates the light-transmissive cavity into the light-transmissive opening; and
a focusing lens disposed on a transmission path of the sensing beam, and disposed between the light source and the light-transmissive opening.

13. The optical sensing module as recited in claim 12, wherein the light-transmissive cavity further includes a reflective layer disposed on an inner surface of the light-transmissive cavity.

14. The optical sensing module as recited in claim 1, wherein the optical sensing module further includes an Analog-to-Digital Converter disposed between the carrying substrate and the optical sensor.

15. The optical sensing module as recited in claim 1, wherein the optical sensing module further includes a shading member disposed around a periphery of the transparent cover, the side wall and the carrying substrate.

16. The optical sensing module as recited in claim 1, wherein a gas pressure of the vacuum chamber has a range that goes from 1×10⁻² torr to 1×10⁻⁷ torr.

17. The optical sensing module as recited in claim 1, wherein the optical sensor is a visible light sensor, an infrared light sensor or an ultraviolet sensor.

18. The optical sensing module as recited in claim 1, wherein the light-transmissive opening is a slit.

19. The optical sensing module as recited in claim 1, wherein the side wall is a gas adsorption layer.

20. The optical sensing module as recited in claim 19, wherein at least one material of the gas adsorption layer includes cooper, aluminium, vanadium, zirconium, cobalt, or a combination thereof.
